(19) United States
(12) Patent Application Publication
(10) Pub. No.: US 2024/0404659 A1
(43) Pub. Date: Dec. 5, 2024

(54) Integrative System and Method for Performing Medical Diagnosis Using Artificial Intelligence

(71) Applicant: AYUR.AI PRIVATE LIMITED, Chennai (IN)

(72) Inventor: Balasubrahmanyam PESALA, Chennai (IN)

(21) Appl. No.: 18/407,184

(22) Filed: Jan. 8, 2024

Related U.S. Application Data
(63) Continuation of application No. PCT/IB22/56265, filed on Jul. 7, 2022.

(30) Foreign Application Priority Data
Jul. 9, 2021 (IN) 202141030804

(51) Int. Cl. G16H 10/60; A61B 5/00; A61B 5/0205; G16H 50/20; G16H 50/30

(52) U.S. Cl. CPC G16H 10/60; A61B 5/0205; A61B 5/7267; A61B 5/7275; G16H 50/20; G16H 50/30

(57) ABSTRACT

An integrative system (100) for performing medical diagnosis using artificial intelligence is disclosed. The integrative system (100) combines traditional medicine with modem medical science. Specifically, a baseline of a patient is first established. Subsequently, changes in the baseline of the patient are captured. Machine learning techniques are incorporated into the integrative system (100) for accurate and quantifiable determination of the baseline of the patient as well as the changes in the baseline that leads to diseases. Further, health, wellness and risk scores are generated based on this integrative approach. The integrative system (100) is also configured to generate a recommendation message to the patient based on the generated health, wellness, and disease risk score. The knowledge from the baseline and baseline changes is used for subsequent interventions such as food, medicine, meditation, yoga, panchakarma, acupuncture, music, massage, and the like.

### EARLIEST PRIORITY DATE

This Application claims priority from a provisional patent application filed in India having patent application Ser. No. 20/214,1030804. filed on Jul. 9, 2021, and titled “SYSTEM AND METHOD FOR INTEGRATIVE COMBINATION OF TRADITIONAL SCIENCE WITH MODERN METHODS FOR PERSONALIZED HEALTH, WELLNESS AND MEDICINE”.

### FIELD OF INVENTION

Embodiments of the present disclosure relates to medical diagnosis systems, and more particularly to a traditional and modern medical science integrative system and a method for performing medical diagnosis using artificial intelligence.

### BACKGROUND

Currently, traditional diagnosis methods of medicine such as Ayurveda, siddha, traditional Chinese medicine, and the like, are gaining popularity due to their proactive nature and ability for deep personalization of diagnosis and treatments. However, these traditional diagnosis methods are highly subjective and expert dependent. Normally, medical professionals fail to have a complete in-depth knowledge of such traditional diagnosis methods of medicine. Furthermore, when traditional methods of medicine are used, no quantifiable metrics for diagnosis are obtained.

On the contrary, modern medical practices such as allopathy are highly reactive in nature. However, such modern medical practices lack personalization as they cannot be structured specifically for any patient. Sometimes, such modern medicine has proven to be not hundred percent effective in curing a disease. Personalization is needed to predict actionable interventions for patients that further improves long-term health and wellness. Actionable interventions as described here may be referred to yoga, medicine, and the like.

Furthermore, to achieve long-term health improvement, a patient illness should be diagnosed and advised with both clinical interventions and therapeutic interventions.

Hence, there is a need for an improved integrative system and method for performing medical diagnosis using artificial intelligence to address the aforementioned issues.

### BRIEF DESCRIPTION

In accordance with one embodiment of the disclosure, an integrative system for performing medical diagnosis using artificial intelligence is disclosed. The integrative system includes the hardware processor. The integrative system also includes a memory coupled to the hardware processor. The memory comprises a set of program instructions in the form of a plurality of subsystems, configured to be executed by the hardware processor.

The plurality of subsystems includes a medical input data collection subsystem. The medical input data collection subsystem is configured to collect patient information and phenotypic features associated with a patient from a plurality of medical devices, computer vision techniques, and from a conversational artificial intelligence questionnaire.

The plurality of subsystems also includes a baseline health computation subsystem. The baseline health computation subsystem is configured to apply the collected patient information and phenotypic features associated with the patient on to a trained machine learning model. The baseline health computation subsystem is also configured to generate baseline health status of the patient based on the results of the application of the trained machine learning model.

The plurality of subsystem also includes a baseline change determination subsystem. The baseline change determination subsystem is configured to obtain a combination of: responses for a set of adaptive and conversational artificial intelligence-based questionnaire in real time from the patient, one or more real time patient information from the plurality of medical devices associated with the patient, one or more real time spatiotemporal information data associated with the patient, and one or more real time inputs from biochemical markers, clinical markers and multi-omics markers.

The baseline change determination subsystem is also configured to estimate real time set of integrative medicine system parameters by applying the obtained responses for the set of adaptive and conversational artificial intelligence-based questionnaire, the obtained one or more real time patient information, the obtained one or more real time spatiotemporal information data and the obtained one or more real time inputs from biochemical markers and multi-omics markers onto the trained machine learning model.

The baseline change determination subsystem is configured to determine changes in the generated baseline health status of the patient by comparing the estimated real time set of integrative medicine system parameters with the estimated set of integrative medicine system parameters associated with the generated baseline health status by using artificial intelligence-based comparison model.

The plurality of subsystems also includes a health, wellness, and disease risk score computation subsystem. The health, wellness, and disease risk score computation subsystem is configured to generate a health, wellness, and disease risk score of the patient based on determined changes in the generated baseline health status of the patient. The health, wellness, and disease risk score computation subsystem is also configured to generate a recommendation message to the patient based on the generated health, wellness, and disease risk score.

In accordance with one embodiment of the disclosure, an integrative method for performing medical diagnosis using artificial intelligence is disclosed. The integrative method includes collecting patient information and phenotypic features associated with a patient from a plurality of medical devices, computer vision techniques, and from a conversational artificial intelligence questionnaire. The integrative method also includes applying the collected patient information and phenotypic features associated with the patient on to a trained machine learning model. The integrative method also includes generating baseline health status of the patient based on the results of the application of the trained machine learning model.

The integrative method also includes collecting and obtaining a combination of: responses for a set of adaptive and conversational artificial intelligence-based questionnaire in real time from the patient, one or more real time patient information from the plurality of medical devices associated with the patient, one or more real time spatiotemporal information data associated with the patient, and one or more real time inputs from biochemical markers, clinical markers and multi-omics markers.

The integrative method also includes estimating real time set of integrative medicine system parameters by applying the obtained responses for the set of adaptive and conversational artificial intelligence-based questionnaire, the obtained one or more real time patient information, the obtained one or more real time spatiotemporal information data and the obtained one or more real time inputs from biochemical markers and multi-omics markers onto the trained machine learning model. The integrative method also includes determining changes in the generated baseline health status of the patient by comparing the estimated real time set of integrative medicine system parameters with the estimated set of integrative medicine system parameters associated with the generated baseline health status by using artificial intelligence-based comparison model.

The integrative method also includes generating a health, wellness, and disease risk score of the patient based on determined changes in the generated baseline health status of the patient. The integrative method also includes generating a recommendation message to the patient based on the generated health, wellness, and disease risk score.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram illustrating an exemplary integrative system for performing medical diagnosis using artificial intelligence in accordance with an embodiment of the present disclosure;

FIG. 2 is a schematic representation of exemplary process depicting integrative approach to determine baseline and baseline changes, in accordance with an embodiment of the present disclosure;

FIG. 3 is a schematic representation depicting an exemplary process of estimating Ayurveda prakriti and vikriti, in accordance with an embodiment of the present disclosure;

FIG. 4 is a schematic representation depicting an exemplary process of estimating Ayurveda vikriti, in accordance with an embodiment of the present disclosure.

FIG. 5 is schematic representation depicting exemplary pulse diagnosis for Ayurveda Vikriti, in accordance with an embodiment of the present disclosure;

FIG. 6 is a schematic representation of smart phone-based approach for exemplary pulse diagnosis and Ayurveda prakriti and vikriti estimation, in accordance with an embodiment of the present disclosure;

FIG. 7 is a process flowchart illustrating an exemplary integrative method for performing medical diagnosis using artificial intelligence in accordance with an embodiment of the present disclosure;

FIG. 8 is a schematic representation depicting process of determining therapeutic interventions from Ayurveda doshas, in accordance with an embodiment of the present disclosure;

FIG. 9A-D are graphical user interface of conversational artificial intelligence based adaptive questionnaire framework designed as per Ayurveda principles for assessing Prakriti baseline of a person, in accordance with an embodiment of the present disclosure;

FIG. 10A-B are snapshot representations of weights of the associations between each symptom to the disease as trained using artificial intelligence techniques such as machine learning, probabilistic reasoning, graph neural networks in accordance with an embodiment of the present disclosure;

FIG. 11A-D are graphical user interface of conversational artificial intelligence-based adaptive questionnaire framework designed as per Ayurveda principles for assessing vikriti of a person, in accordance with an embodiment of the present disclosure; and

FIG. 12A-C are graphical user interface of conversational artificial intelligence-based representation for Ayurveda recommendation regarding infectious diseases such as COVID based on the prakriti and vikriti assessment in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “subsystem” that is configured and operated to perform certain operations. In one embodiment, the “subsystem” may be implemented mechanically or electronically, so a subsystem may comprise dedicated circuitry or logic that is permanently configured (within a special-purpose processor) to perform certain operations. In another embodiment, a “subsystem” may also comprise programmable logic or circuitry (as encompassed within a general-purpose processor or other programmable processor) that is temporarily configured by software to perform certain operations.

Accordingly, the term “subsystem” should be understood to encompass a tangible entity, be that an entity that is physically constructed permanently configured (hardwired) or temporarily configured (programmed) to operate in a certain manner and/or to perform certain operations described herein.

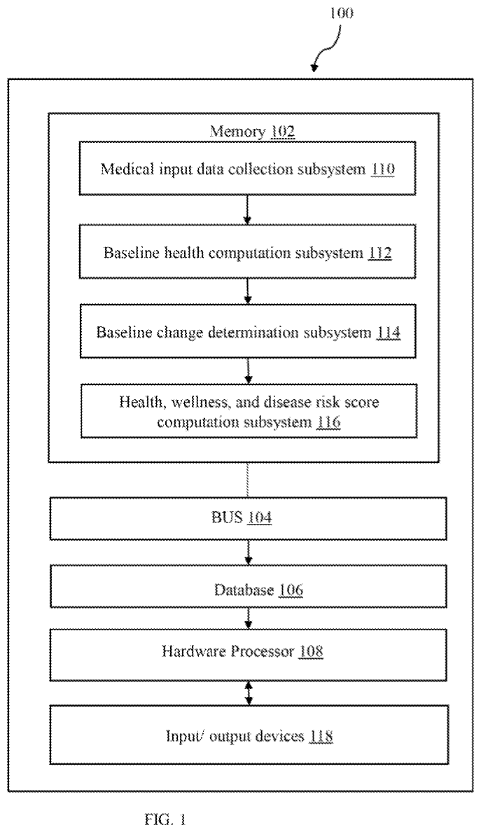

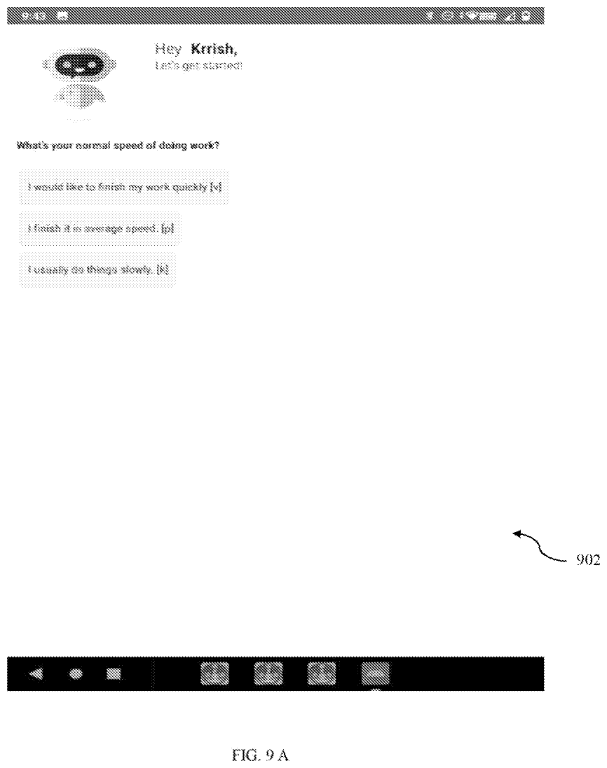

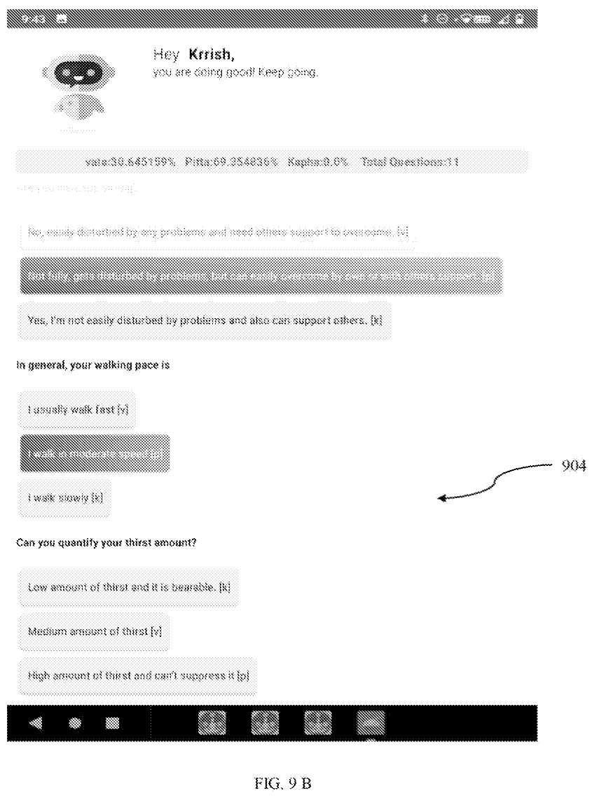

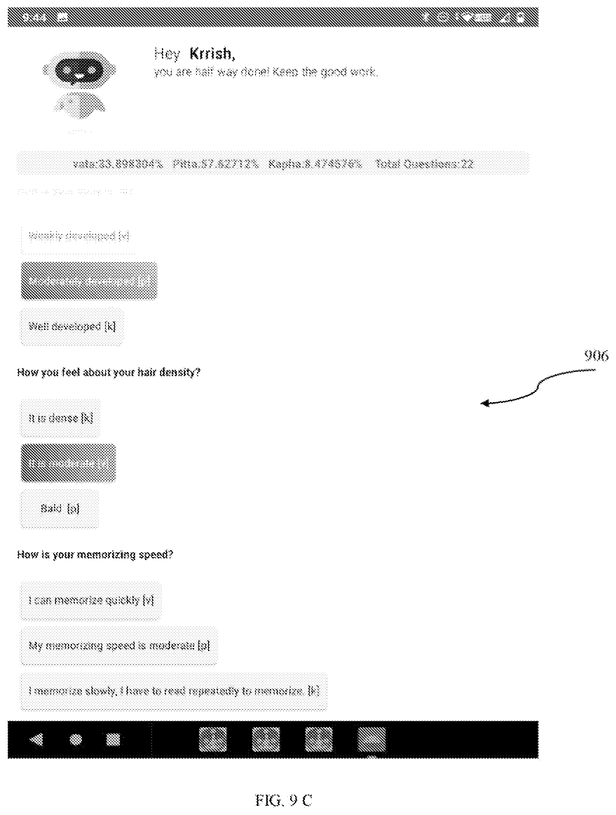

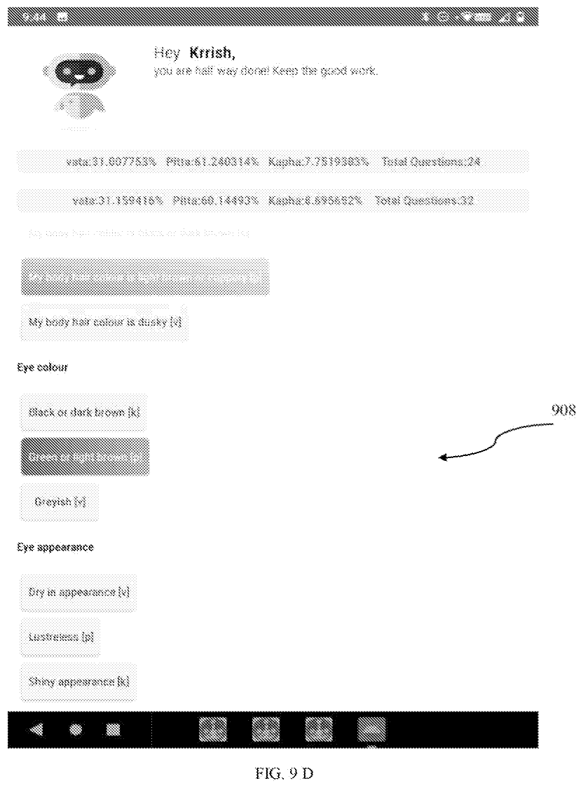

FIG. 1 is a block diagram illustrating an exemplary integrative system 100 for performing medical diagnosis using artificial intelligence in accordance with an embodiment of the present disclosure. The integrative system 100 combines traditional medicinal sciences such as Ayurveda, traditional Chinese medicine, Siddha, Unani, and the like, with modern medical science for highly personalized and quantifiable medical diagnosis. The integrative system 100 recommends actionable interventions such as food, medicine, yoga, music, mantras/chantings, nutraceuticals, medicine, treatment procedures and the like. This is shown in FIG. 9.

The integrative system 100 includes the hardware processor 108. The integrative system 100 also includes a memory 102 coupled to the hardware processor. The memory 102 comprises a set of program instructions in the form of a plurality of subsystems, configured to be executed by the hardware processor 108.

The hardware processor(s) 108, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 102 includes a plurality of subsystems stored in the form of executable program which instructs the hardware processor 108 via bus 104 to perform the method steps. Bus 104 enables communication between all memory 102, hardware processor 108 and database 106. Input/output (I/O) devices 118 (including but not limited to keyboards, displays, pointing devices, etc.) can be coupled to the system either directly or through intervening I/O) controllers. The plurality of subsystems of has following subsystems: a medical input data collection subsystem 110, a baseline health computation subsystem 112, baseline change determination subsystem and a health 114, wellness, and disease risk score computation subsystem 116.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 108.

The plurality of subsystems includes a medical input data collection subsystem 110. The medical input data collection subsystem 110 is configured to collect patient information and phenotypic features associated with a patient from a plurality of medical devices, computer vision techniques, and from a conversational artificial intelligence questionnaire.

The phenotypic features include anatomic features, physical, physiological features and psychological features. The anatomic features include appearance features such as body dimensions, face symmetry, hair and eye colour, skin texture, and the like. Physical features include walking speed, voice quality, speech attributes, speaking speed, and the like. Physiological features sleep quality, digestion details, food preferences, weather preferences, and the like. Psychological features include anger & irritability, cognitive abilities, and the like.

In one embodiment, the phenotypic feature includes features extracted from pulse electrocardiography (ECG) data, photo plethysmograph (PPG) data, electroencephalogram (EEG) data, bioimpedance sensor data, galvanic skin response data, multispectral reflectance data, transmittance data, and autofluorescence data from various body parts, sleep activity data, physical activity data and mental activity data, and the like. In such embodiment, the plurality of medical devices includes various medical equipment, smart devices and the like. For example, a smart watch may be used to measure and collect data related to patient blood pressure, patient blood oxygen level, patient heart rate, patient pulse waveform, and the like.

In another embodiment, the anatomic features of the patient as described, are extracted using computer vision techniques; and digital biomarkers (both static markers while the patient is at rest and also dynamic markers that are captured as per the patient transitions from one state to other (such as from sitting to standing or lying to standing, walking up the stairs etc.) are obtained using various physiological sensors, voice-based techniques and various biochemical, clinical and multi-omics markers. Multi-omics markers include genetic, transcriptome, epigenome, metagenome, metabolome, glycome, proteome and the like. Digital biomarkers are defined as objective, quantifiable physiological and behavioural data that are collected and measured by means of digital devices such as portables, wearables, implantable, or digestible.

In yet another embodiment, the conversational artificial intelligence questionnaire is used for capturing patient information and phenotypic features. The patient information includes age of the patient, gender of the patient, family history of the patient and the like. The phenotypic features include the anatomic features, physical and physiological features, psychological features, and the like. The conversational artificial intelligence questionnaire is presented to the patient in a digital format such as computers, phones, tablets, smart watches, and the like. In such embodiment, the conversational artificial intelligence questionnaire may have both text and voice format. The conversational artificial intelligence questionnaire also has multilingual support. In such embodiment, the integrative system 100 optimizes the number and (or) type of questions needed to assess the baseline based on replies given to the previous questions until a desired accuracy level is reached.

The plurality of subsystems also includes a baseline health computation subsystem 112. The baseline health computation subsystem 112 is configured to apply the collected patient information and phenotypic features associated with the patient on to a trained machine learning model.

For example, to create a baseline health status for the patient's heart condition, the integrative system 100 collects readings corresponding to the physiological condition of the patient from the plurality of medical devices. The computer system 100 also collects data regarding anatomic features and other phenotypic features using computer vision techniques of the patient to arrive at various digital biomarkers.

A questionnaire response for the conversational artificial intelligence questionnaire is also collected by the integrative system 100. In such exemplary embodiment, the collected readings corresponding to the heart condition are combined to determine the baseline health status of the patient's heart condition. The questionnaire may include genetic history of various diseases in the family, death due to heart attacks, behaviour inputs such as type of food eaten, increase in weight over the years, exercise frequency and clinical symptoms such as pain in the chest, dizziness after exercise, and the like. In such embodiment, the collected readings are combined with health information of the patient.

The trained machine learning model uses algorithms such as logistic regression, random forests, support vector machines, nearest neighbour clustering, boosted generalized additive models, neural networks, and the like. or may include a combination of the above algorithms. In one embodiment, the conversational artificial intelligence questionnaire is converted into categorical variables in a matrix form and then applied to the trained machine learning model to arrive at a baseline health status. In such embodiment, the other forms of collected data is directly applied to the trained machine learning model.

The baseline health computation subsystem 112 is also configured to generate baseline health status of the patient based on the results of the application of the trained machine learning model. The generated baseline health status comprises a set of integrative medicine system parameters of the patient. The set of integrative medicine system parameters comprise a set of traditional medicine system and a set of modern medicine system parameters of the patient. In one embodiment, the set traditional medicine system parameters include at least one of Ayurveda variables, traditional Chinese medicine variables, Siddha parameters, Unani parameters, and the like.

In one embodiment, the set of Ayurveda variables refers to Ayurveda vata variable, pitta variable and kapha variable. In one embodiment, the Ayurveda vata, pitta and kapha variable baseline is generated based on the generated baseline health status from output of the trained machine learning model. The estimated set of Ayurveda variables are stored in a database 106.

In another embodiment, the baseline health computation subsystem 112 is also configured to estimate a set of Traditional Chinese Medicine (TCM) variables associated with the generated baseline health status based on the result of the application of the trained machine learning model. The result of application of the trained machine learning model is output of the trained machine learning model. In one embodiment, the set of Traditional Chinese Medicine variables refer to Traditional Chinese Medicine Cun variable, Guan variable and Chi variable. In another embodiment, the Traditional Chinese Medicine Cun variable, Guan variable and Chi variable baseline is generated based on the generated baseline health status from output of the trained machine learning model. The estimated set of Traditional Chinese Medicine variables are stored in a database 106.

The plurality of subsystem also includes a baseline change determination subsystem 114. The baseline change determination subsystem 114 is configured to obtain a combination of: responses for a set of adaptive and conversational artificial intelligence-based questionnaire in real time from the patient, one or more real time patient information from the plurality of medical devices associated with the patient, inputs from the computer vision techniques, one or more real time spatiotemporal information data associated with the patient, and one or more real time inputs from biochemical markers, clinical markers and multi-omics markers. In such embodiment, the real time data is collected to understand current health condition of the patient.

In one embodiment, the one or more real time spatiotemporal information associated with the patient includes health parameters of the patient with respect to a particular location and a period of time. As used herein, the term “Spatiotemporal, or spatial temporal” is used in data analysis when data is collected across both space and time. Example of spatial information includes changes in physiological health variables such as heart rate, blood oxygenation, blood pressure of a person at different locations due to varying geographical conditions such as temperature, humidity, weather, altitude, and the like. Examples of temporal information includes changes in the physiological health variables during the time of the day, season, years or changes due to psychological or physical workloads.

The baseline change determination subsystem 114 is also configured to estimate real time set of integrative medicine system parameters by applying the obtained responses for the set of adaptive and conversational artificial intelligence-based questionnaire, the obtained one or more real time patient information, inputs from the computer vision techniques, the obtained one or more real time spatiotemporal information data and the obtained one or more real time inputs from biochemical markers and multi-omics markers to a trained machine learning or deep learning model. Each of the above inputs is first converted into a categorical variable and passed onto a trained model. The trained model automatically identifies the risk score for a particular set of diseases. The diseases may also include sub-types as described in the traditional medicine.

In one exemplary embodiment, to determine the current health condition of the patient, the integrative system 100 collects information corresponding to adaptive questioning, the one or more real time device data, the one or more real time outputs from biochemical markers, clinical markers and multi-omics markers and the one or more real time spatiotemporal information data. In such exemplary embodiment, the collected information is applied to the trained machine learning model to estimate real time set of integrative medicine system parameters. The set of integrative medicine system parameters comprise a set of traditional medicine system and a set of modem medicine system parameters of the patient. In one embodiment, the set of traditional medicine system parameters includes Ayurveda doshas such as vata, pitta and kapha.

The baseline change determination subsystem 114 is configured to determine changes in the generated baseline health status of the patient by comparing the estimated real time set of integrative system parameters with the estimated set of integrative system parameters associated with the generated baseline health status by using artificial intelligence-based comparison model. The artificial intelligence model may use absolute or root mean square, or % changes in the integrative medicine system parameters and use such stated inputs to the model such as logistic regression, random forests, support vector machines, nearest neighbour clustering, boosted generalized additive models to arrive at a risk score corresponding the change in baseline health status of an individual.

In one embodiment, the baseline change determination subsystem 114 is configured to evaluate each of the estimated set of integrative system parameters associated with the generated baseline health status and each of the estimated real time set of integrative system parameters. Simultaneously, each of the estimated set of integrative system parameters associated with the generated baseline health status is compared with each of the estimated real time set of integrative system parameters to determine real time changes. In such embodiment, the artificial intelligence-based comparison model may include deep learning network. The absolute or root mean square, or % changes in the traditional medicine system and modern system parameters and various spatial and time trajectories of the traditional medicine system and modern medical system parameters are used as inputs to the deep learning model to arrive at a risk score corresponding the change in baseline health status of an individual.

The plurality of subsystems also includes a health, wellness, and disease risk score computation subsystem 116. The health, wellness, and disease risk score computation subsystem 116 is configured to generate a health, wellness, and disease risk score of the patient based on determined changes in the generated baseline health status of the patient. The health, wellness, and disease risk score computation subsystem 116 uses various inputs from baseline health status and changes in the baseline health status and uses trained machine learning/deep learning algorithms. The algorithms subsequently generate health and wellness score by comparing the baseline changes with the baseline changes of a set-of-healthy population. The disease risk scores compare the symptoms, inputs from the digital sensors, biochemical and multi-omics markers of the individual with a set of inputs from the diseased population and gives probabilities of risk corresponding to diseases.

The health, wellness, and disease risk score computation subsystem 116 is also configured to generate a recommendation message to the patient based on the generated health, wellness, and disease risk scores. In such embodiment, the recommendation message comprises medical diagnosis of the disease, health parameters, therapeutic or clinical interventions, one or more medical remedies, and treatment plan. The clinical interventions may use additional tests to improve the diagnostic and recommendations accuracy. The therapeutic/clinical interventions may include food, medicine, meditation, music, panchakarma, acupuncture and the like. The recommendation message uses a prior knowledge from the traditional medicine and (or) clinical evidence to suggest a personalized recommendation.

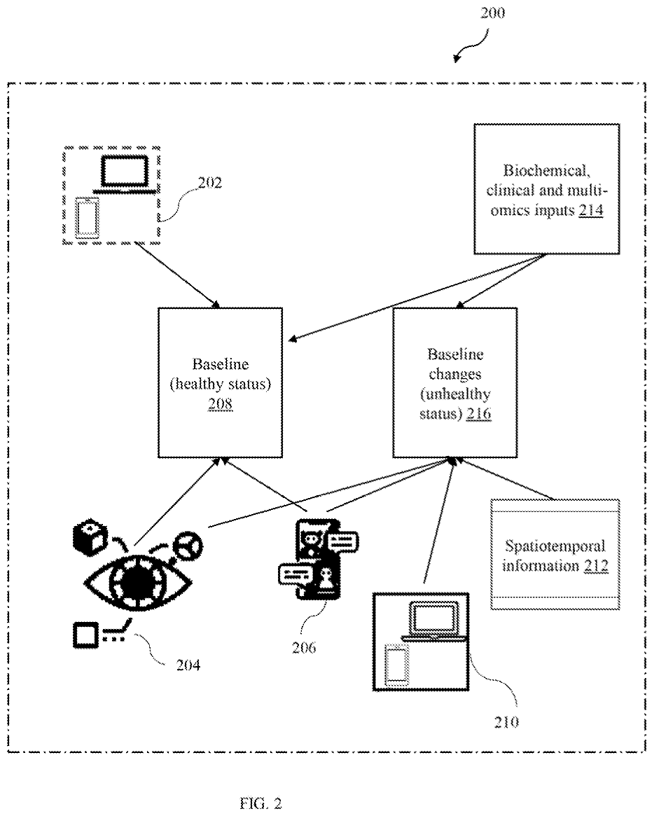

FIG. 2 is a schematic representation of exemplary process depicting integrative approach 200 to determine baseline 208 and baseline changes 216, in accordance with an embodiment of the present disclosure.

A baseline 208 depicting a health state of the patient is generated using inputs from digital devices 202, biochemical, clinical, multi-omics markers 214 and computer vision techniques 214. The computer vision techniques 204 are used to capture anatomic, physiological features and the digital devices 202 are used for determining physiological parameters, sleep activities, eating patterns and the like of the patient. Further, certain biochemical, clinical, multi-omics markers 214 are also used to establish the baseline of the patient.

For the baseline 208 estimation of the patient, a set of questionnaires 206 is also used in combination (as shown in FIG. 9A-D). The set of questionnaires 206 is used for capturing patient information and phenotypic features. The patient information includes age of the patient, gender of the patient, family history of the patient and the like. The phenotypic features include the anatomic features, physical and physiological features, psychological features, and the like. The set of questionnaires 206 may be presented to the patient in a digital format such as computers, phones, tablets, smart watches, and the like.

The input from the set of questionnaires 206 is combined with the inputs from the digital device 202 and biochemical, clinical and multi-omics markers 204. The integrative system 200 determines an accurate baseline 208 of the patient health.

Further, a change in baseline 216 of the patient is established. The inputs are captured from various digital medical devices 210, computer vision techniques 204 and biochemical and multi-omics markers 214. The digital medical devices 210 capture various physiological parameters such as electrocardiography (ECG), photo plethysmograph (PPG), electroencephalogram (EEG), sleep activity, physical and mental activities, eating patterns, environmental parameters, and the like of the patient. In addition, the digital medical devices 210 may also capture multispectral reflectance, multispectral transmittance, multispectral autofluorescence using multispectral sensors (such as LEDs and detectors) from various body parts to understand various key parameters such as blood glucose and the like. Both static and dynamic digital biomarkers are captured to accurately establish the baseline changes.

The biochemical, clinical and multi-omics 214 input includes blood tests, genome, exome, transcriptome, metabolome, epigenome, microbiome, proteome, glycome, and the like. The spatiotemporal information 212 is also collected in real time which includes information such as time of the day of symptoms, type of season, type of weather, geographical location of the patient, information on the gender and menstruation cycle, patient history and the like.

The input from the set of questionnaires 206 is combined with the inputs from the digital medical devices 210, and the biochemical, clinical, and multi-omics 214 inputs to determine the changes in the baseline 216. The spatiotemporal information 212 is also combined to understand the changes in the baseline 216. The baseline 208 and the changes in the baseline 216 are compared using artificial intelligence-based comparison model to generate a health, wellness, disease risk-score. The artificial intelligence-based comparison model comprises machine learning and deep learning techniques. The health, wellness, disease risk-score indicates the health status of the patient in terms of traditional medicine variables such as Ayurveda variables which is Vata, Pitta and Kapha, Ayurveda diseases, Traditional Chinese medicine variable such as Cun, Guan and Chi, Traditional Chinese medicine diseases and (or) integrative system variables such as cardiovascular score, renal score, metabolic score, biological aging score etc. The health, wellness, disease risk-score indicates diagnosis of Roga (or) diseases of the patient and thus helps generate a suitable recommendation message to the patient. The health, wellness, disease risk score is a number ranging between, for example, 0 to 100, where 0 indicates least score and 100 indicates highest scoring and better health condition.

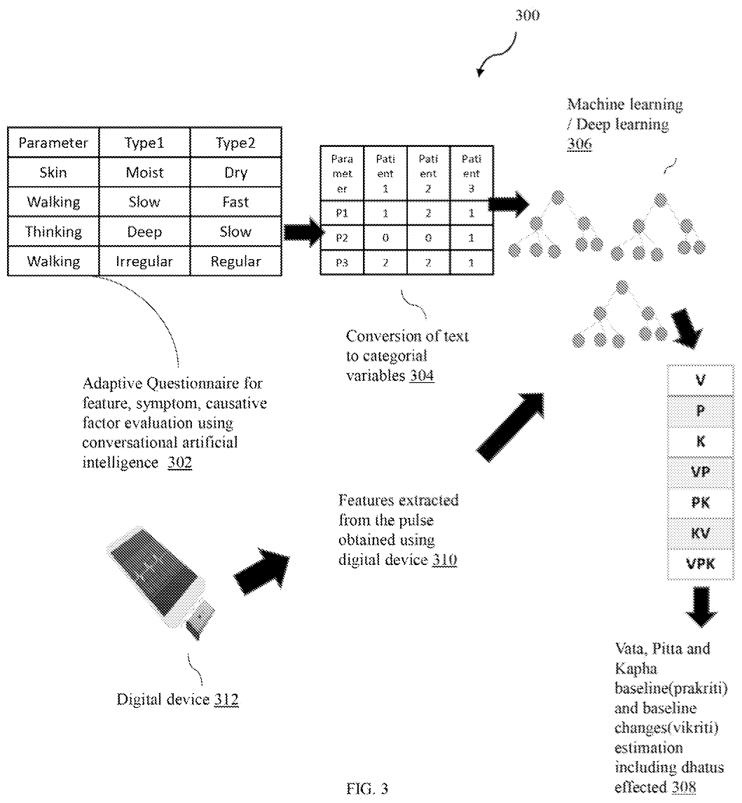

FIG. 3 is a schematic representation depicting an exemplary process of estimating Ayurveda prakriti and vikriti 300, in accordance with an embodiment of the present disclosure. As used herein, the term “feature” refers to different phenotypic parameters of the patient, such as physical, psychological, anatomical, physiological, for example, skin colour, digestion, thinking, walking and the like. In another exemplary embodiment, these adaptive questionnaires 302 comprises health symptom-based questions, causative factors for symptoms in text format. In such embodiment, the adaptive questionnaires 302 comprises questions related to disease symptoms such as pain in certain part of the body, sleep disorders, changed body weight, excessive thirst or urination and the like. The adaptive questionnaire may also include questions related to causative factors for the symptoms such as eating certain foods, exposure to environmental factors dust, infections etc., physical and mental stress, consumption of certain medication etc. These adaptive questionnaires 302 are outputted to the patient on his device for obtaining a response for each of these adaptive questionnaires 302.

The text of the questionnaire is converted into categorical variables 304, such as 0s, 1s and 2s. Furthermore, trained machine learning models 306 are used onto each of these categorical variables 304 to estimate the set of Ayurveda variable, Vata, Pitta and Kapha 308. In one exemplary embodiment, a random forest tree machine learning algorithm may be used.

Simultaneously, inputs from digital devices 312 are collected. Then, features 310 are extracted from pulse obtained using the digital device 312. In such embodiment, the digital device 312 may be a wearable device, such as a smartwatch. The digital device may also be a smart phone. These extracted features 310 comprises pulse rate, pulse rate variability, nonlinear measures of pulse rate variations, pulse pressure, pulse morphology, and the like. are also fed as additional input to the trained machine learning models 306 to estimate the set of Ayurveda variables, baseline Vata, Pitta and Kapha.

The output of the machine learning model 306 is used for estimating Ayurveda vata, pitta and kapha baseline and changes in vata, pitta and kapha with respect to the baseline positions of the patient 308. The changes of vata, pitta and kapha with respect to the baseline vata, pitta and kapha forms Vikriti. In addition, the dhatus (as defined in Ayurveda such as blood, muscle, fat, plasma, bone, bone marrow etc.) effected due to the baseline changes in vata, pitta, kapha are also estimated.

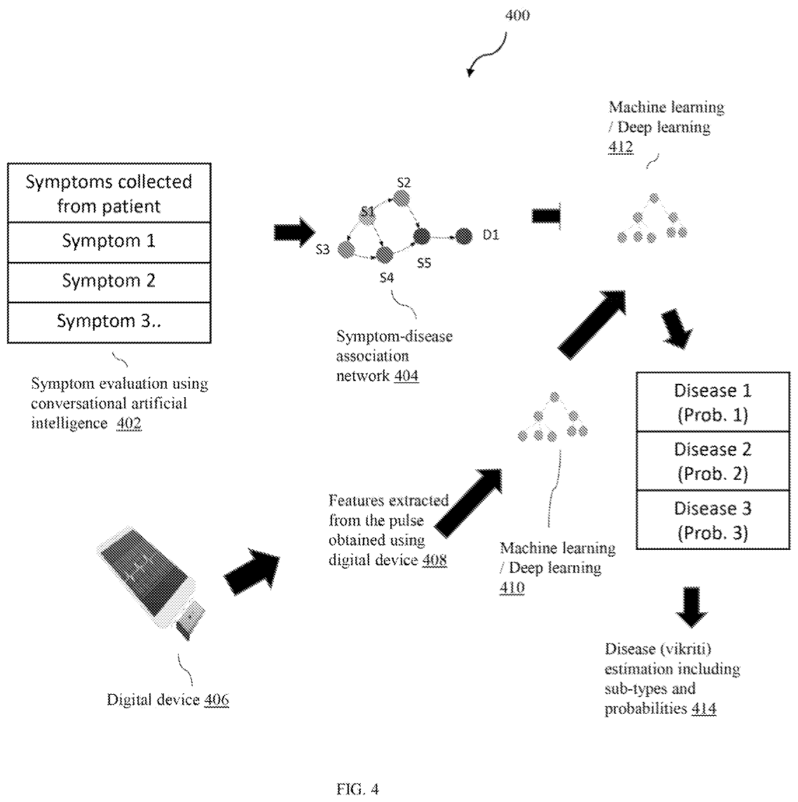

FIG. 4 is a schematic representation depicting an exemplary process of estimating Ayurveda vikriti, in accordance with an embodiment of the present disclosure. In such embodiment, the adaptive questionnaires 402 based on conversational artificial intelligence comprises questions related to disease symptoms such as pain in certain part of the body, sleep disorders, changed body weight, excessive thirst or urination, and the like. The questionnaire is adaptive where the next question to understand the presence or absence of a particular symptom is presented based on response to the previous symptom. In a particular embodiment, the adaptive questionnaire 402 is designed based on the symptom-disease association network 404. These adaptive questionnaires 402 are outputted to the patient on his device for obtaining a response for each of these adaptive questionnaires 402. The adaptive questionnaire helps in triaging the possible diseases of the patient based on the symptoms.

The responses of the symptom questionnaire are compared with the symptom-disease association network 404. The symptom-disease association network 404 may be trained on various machine learning, deep learning models. In one exemplary embodiment, a graph neural network may be used for outputting the diseases along with probabilities corresponding to particular symptoms.

Simultaneously, inputs from digital devices 406 are collected. Then, features 408 are extracted from pulse obtained using the digital device. In such embodiment, the digital device 406 may be a smart phone device with inbuilt sensors such as accelerometer, gyrometer, camera etc. or attached with various external sensors such as ECG, PPG etc. The device is used to extract 408 features such as pulse rate, pulse rate variability, nonlinear measures of pulse rate variations, pulse pressure, pulse morphology, and the like, are also fed as additional input to the trained machine learning models 410 to estimate the possible diseases along with explainable artificial intelligence framework in terms of the symptoms contributing to identification of a particular disease along with the probabilities of the diseases.

The outputs from 404 and 410 are given as inputs to another machine or deep learning algorithm 412. The trained algorithm 412 aggregates the inputs and gives a decision on the Ayurveda diseases including disease sub-types with probabilities 414.

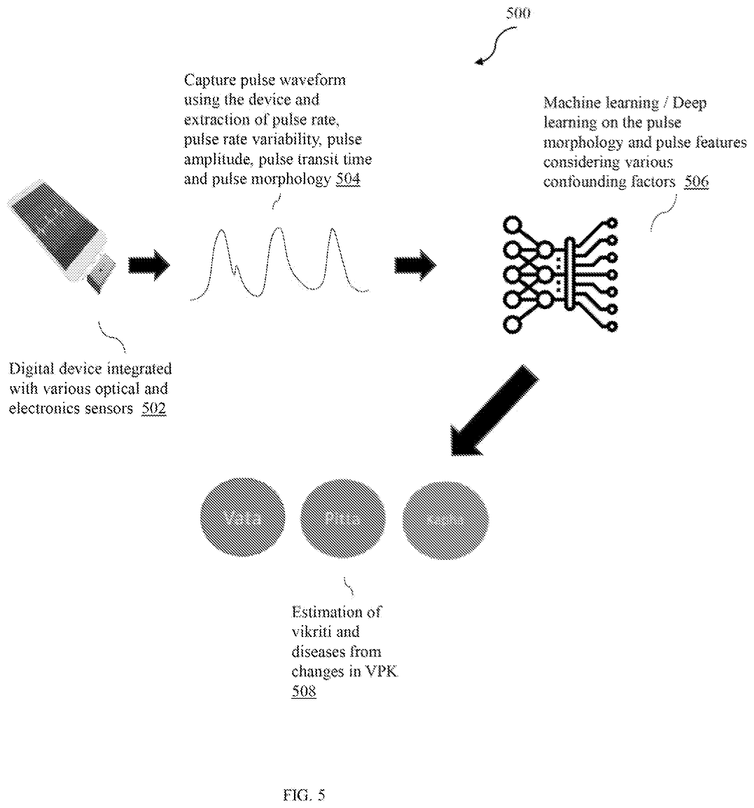

FIG. 5 is schematic representation depicting exemplary pulse diagnosis for Ayurveda Vikriti 500, in accordance with an embodiment of the present disclosure. A digital device such as a smart phone 502 is integrated with various optical and electronics sensors such as photo plethysmography, electrocardiography, accelerometer, barometer, Bioimpedance, Galvanic skin response, and the like. In another such embodiment, the digital device may be a wearable device, handheld device, tablets and the like.

In such embodiment, such digital devices with inbuilt sensor 502 capture a pulse waveform 504 of the patient. In addition, inputs from the sensors in the digital device such as accelerometer, gyrometer etc. are used to compensate for various noises. Pulse features such as pulse rate variability, pulse amplitude, pulse transit time and pulse morphology are extracted from the pulse waveform 504. Here, pulse morphology refers to pulse shape which captures the amplitude changes of the pulse with respect to time. A trained machine learning model 506 is used on the extracted pulse features and pulse morphology and also considers various confounding factors such as time of the day, season, weather, sleep habits, stress levels etc captured using the digital device. The output of the machine learning techniques generates estimation of Vikriti based on changes in vata, pitta and kapha 508.

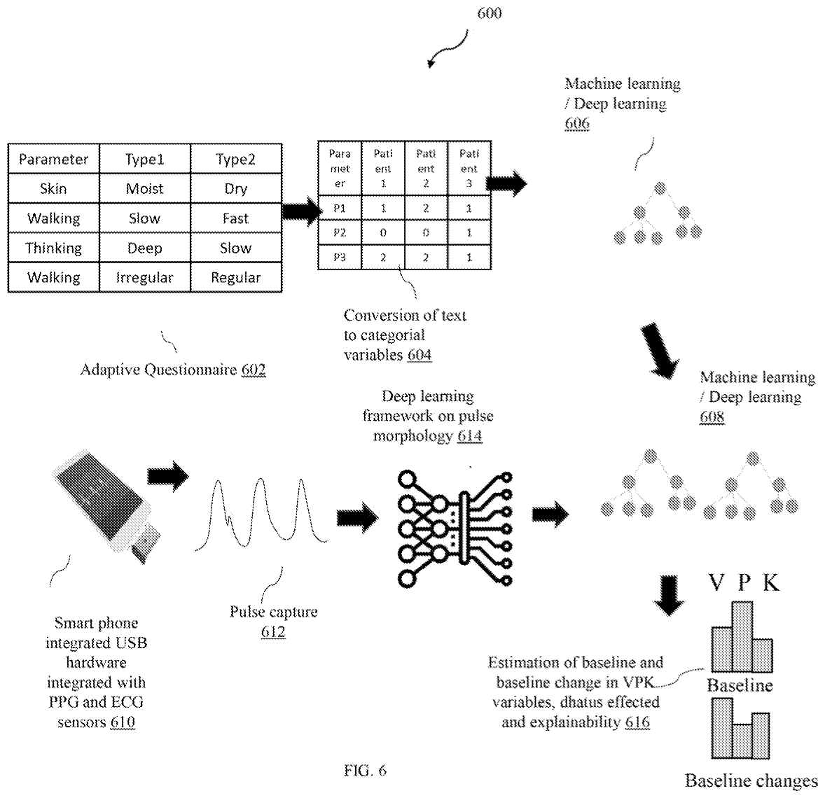

FIG. 6 is a schematic representation of smart phone-based approach 600 for Ayurveda diagnosis and Ayurveda prakriti and vikriti estimation, in accordance with an embodiment of the present disclosure. In one embodiment, responses for a set of adaptive and conversational artificial intelligence-based questionnaire 602 are collected. The adaptive and conversational artificial intelligence-based questionnaire 602 may include questions related to phenotypic characteristics of the patient such as eyes, ears, hair, hands, and the like. These adaptive and conversational artificial intelligence-based questionnaires 602 are in text format. The response of the adaptive and conversational artificial intelligence-based questionnaire 602 is converted to categorical variables 604 such as 0s, 1s and 2s in a matrix form.

In such embodiment, supervised machine learning algorithm 606 is applied to estimate vata, pitta and kapha. The outputs of this machine learning algorithm are given as inputs to another machine learning algorithm 608 for baseline and baseline changes in vata, pitta and kapha and explainability of the decision of the machine learning algorithm is given for easy interpretation of the results 616.

Simultaneously, a digital device such as smart phone 610 is included. The smart phone 610 is integrative with a universal serial bus (USB) hardware which is integrated with photo plethysmograph (PPG), Electrocardiogram (ECG) sensors and Galvanic skin response (GSR) sensors. Alternatively, the camera of the smartphone 610 may be used to collect the photo plethysmograph data. This smart phone 610 captures physiological parameters such as blood pulse 612 of the patient. In addition, inputs from the sensors in the digital device such as accelerometer, gyrometer etc. are used to compensate for various noises. The pulse capture 612 is applied to a trained deep learning model 614. The model also considers various confounding factors such as time of the day, season, weather, sleep habits, stress levels etc captured using the digital device. The outputs from the trained deep learning model 614 are further given to machine/deep learning algorithm 608 for baseline and baseline changes in vata, pitta and kapha variables, dhatus effected and explainability in terms of features contributing to the baseline and baseline changes are given for easy interpretation of the results 616.

In an exemplary embodiment, the integrative system 100 provides a web-based Ayurveda application deployable either independently or together onto multiple devices such as a smart phone, smart computer, smart tablet, a smart band and a digital kiosk and the like.

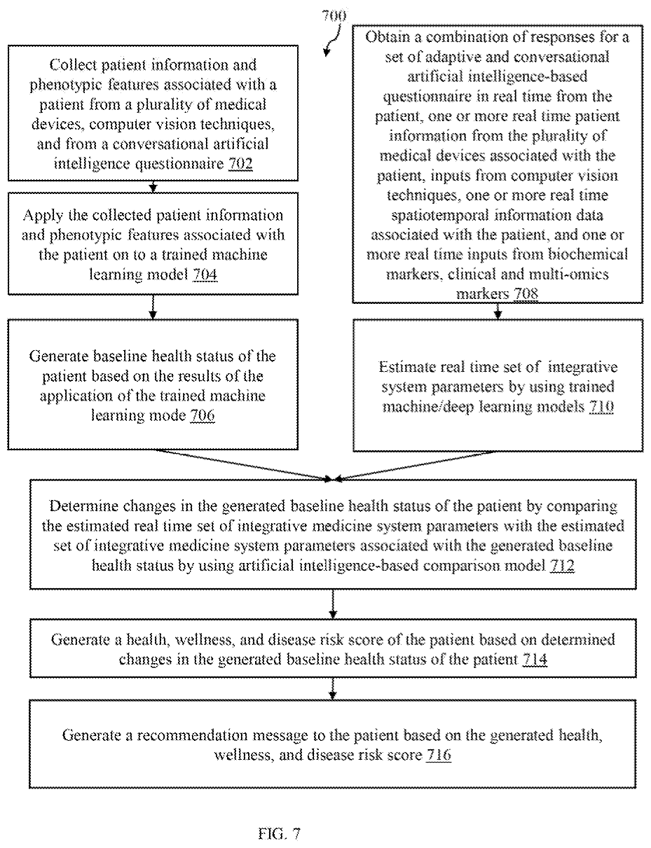

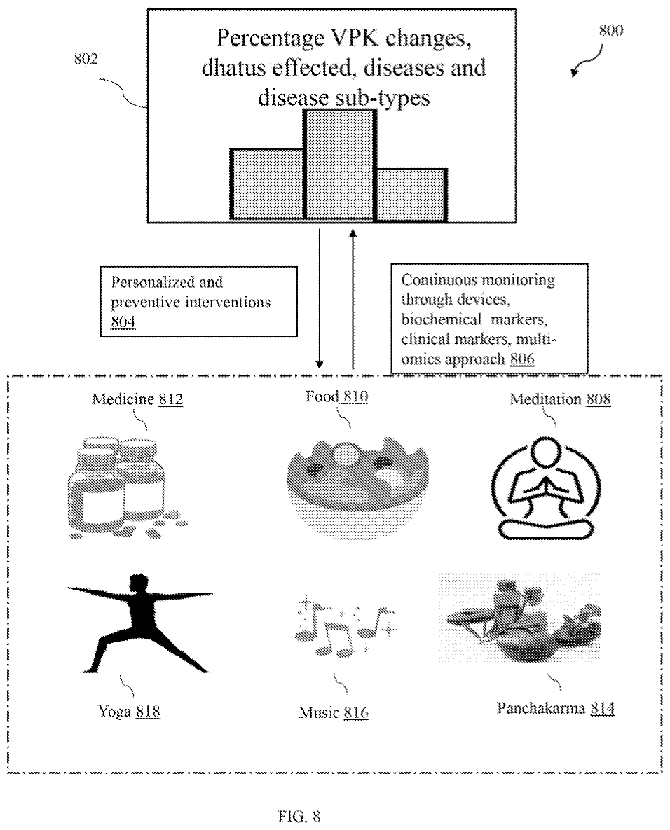

FIG. 7 is a process flowchart illustrating an exemplary integrative method 700 for performing medical diagnosis using artificial intelligence in accordance with an embodiment of the present disclosure. At step 702, patient information and phenotypic features associated with a patient are collected from a plurality of medical devices 202, computer vision techniques 204, and from a conversational artificial intelligence questionnaire 206. In one embodiment, the patient information and the phenotypic features associated with the patient from the plurality of medical devices 202, computer vision techniques 204, and from a conversational artificial intelligence questionnaire 206 are collected by a medical input data collection subsystem 110.

In one specific embodiment, the phenotypic features comprise pulse electrocardiography (ECG) data, photo plethysmograph (PPG) data, electroencephalogram (EEG) data, bioimpedance sensor data, galvanic skin response data, multispectral reflectance data, transmittance data, and autofluorescence data from various body parts, sleep activity, physical and mental activities. In another specific embodiment, in collecting the anatomic features of the patient, the method 700 comprises obtaining features using various physiological sensors, voice-based techniques and various biochemical, clinical and multi-omics markers.

In yet another specific embodiment, the conversational artificial intelligence questionnaire 206 comprises capturing of patient information and phenotypic features. The phenotypic features include anatomical features, physical, physiological features, psychological features. The patient information includes age of the patient, gender of the patient, family history of the patient.

At step 704, the collected patient information and phenotypic features associated with the patient is applied on to a trained machine learning model. In one aspect of the present embodiment, the collected patient information and phenotypic features associated with the patient is applied by the baseline health computation subsystem 112.

At step 706, baseline health status of the patient is generated based on the results of the application of the trained machine learning model. In one aspect of the present embodiment, the baseline health status of the patient is generated based on the results of the application of the trained machine learning model by the baseline health computation subsystem 112. The generated baseline health status comprises a set of traditional medicine system parameters and modern medicine system parameters of the patient. In such embodiment, the set of traditional medicine system parameters comprises Ayurveda variables or doshas, traditional Chinese medicine variables, Unani variables and the like. The set of modern medicine system parameters include cardiovascular fitness score, metabolic score, biological age, respiratory fitness score and the like.

At step 708, a combination of: responses for a set of adaptive and conversational artificial intelligence-based questionnaire in real time from the patient, one or more real time patient information from the plurality of medical devices associated with the patient, one or more real time spatiotemporal information data associated with the patient, and one or more real time inputs from biochemical markers and multi-omics markers are obtained. In one aspect of the present embodiment, the combination of: responses for the set of adaptive and conversational artificial intelligence-based questionnaire in real time from the patient, inputs from the computer vision techniques, the one or more real time patient information from the plurality of medical devices associated with the patient, the one or more real time spatiotemporal information data associated with the patient, and the one or more real time inputs from biochemical markers, clinical markers and multi-omics markers are obtained by the baseline change determination subsystem 114. In such embodiment, the one or more real time spatiotemporal information data associated with the patient includes health parameters of the patient with respect to a particular location and period of time.

At step 710, real time set of integrative medicine system parameters are estimated by applying the obtained responses for the set of adaptive and conversational artificial intelligence-based questionnaire, the obtained one or more real time patient information, the obtained one or more real time spatiotemporal information data and the obtained one or more real time inputs from biochemical markers and multi-omics markers onto a trained machine learning model. In one aspect of the present embodiment, the real time set of integrative medicine system parameters are estimated by the baseline change determination subsystem 114.

At step 712, changes in the generated baseline health status of the patient are determined by comparing the estimated real time set of integrative medicine system parameters with the estimated set of traditional medicine system and modern medicine system parameters associated with the generated baseline health status by using artificial intelligence-based comparison model. In one aspect of the present embodiment, the changes in the generated baseline health status are determined by the baseline change determination subsystem 114.

In such embodiment, in comparing the estimated real time set of integrative medicine system parameters and the estimated set of integrative medicine system parameters associated with the generated baseline health status, the method 700 comprises comparing of each of the estimated set of integrative medicine system associated with the generated baseline health status and each of the estimated real time set of integrative medicine system parameters.

Furthermore, after evaluation, each of the estimated set of integrative medicine system parameters associated with the generated baseline health status is compared with each of the estimated real time set of integrative medicine system parameters to determine real time changes.

At step 714, a health, wellness, and disease risk score of the patient is generated based on determined changes in the generated baseline health status of the patient. In one aspect of the present embodiment, the health, wellness, and disease risk score of the patient is generated based on the determined changes in the generated baseline health status of the patient by a health, wellness, and disease risk score computation subsystem 116.

At step 716, a recommendation message is generated to the patient based on the generated health, wellness, and disease risk score. In one aspect of the present embodiment, the recommendation message is generated to the patient based on the generated health, wellness, and disease risk score by the health, wellness, and disease risk score computation subsystem 116. In such embodiment, the recommendation message to the patient comprises medical diagnosis of the disease, health parameters, therapeutic interventions, clinical interventions, one or more medical remedies, and treatment plan.

FIG. 8 is a schematic representation of depicting process 800 of determining therapeutic interventions from Ayurveda doshas in accordance with an embodiment of the present disclosure. From the estimated Ayurveda doshas 802 such as changes in vata, pita, kapha (VPK), dhatus effected, Ayurveda diseases including disease sub-types, the personalized and preventive interventions 804 are determined. The therapeutic interventions include food 810, medicine 812, meditation 808, yoga 818, music 816, panchakarma 814 and the like. In such embodiment, the whole intervention process is continuously monitored 806 through devices, biochemical markers, clinical markers and multi-omics approach. In such embodiment, supervised or un-supervised machine learning and deep learning techniques are applied to collect patient information and also to extract digital biomarkers. therapeutic interventions are tailored according to such monitored output.

FIG.9A-D are graphical user interface of conversational artificial intelligence based adaptive questionnaire framework designed as per Ayurveda principles for assessing Prakriti baseline of a person 902, 904, 906 and 908, in accordance with an embodiment of the present disclosure. Each of the question probes the Vata, Pitta or Kapha characteristics of the person based on the phenotypic features. The weights for each of the questions are pre-assigned based on the clinical knowledge or from the machine learning algorithm. At the end of each question/set of questions, the scores are calculated for Vata, Pita and Kapha.

FIG. 9B, C and D shows the calculated scores of Vata, Pita and Kapha. The questions are presented to the subjected till the successive difference in the scores from a question/set of questions is less than a pre-set threshold value. This conversational artificial intelligence based adaptive questionnaire saves significant time in assessing the prakriti (baseline) of any person while achieving the desired accuracy. Some of the questions pertaining to prakriti estimation are “how you feel about your hair density?”, “What's your normal speed of doing work?”, “eye colour?”, and the like. Similar conversational artificial intelligence-based framework may also be used for assessing the Vikriti (diseased state) of a person.

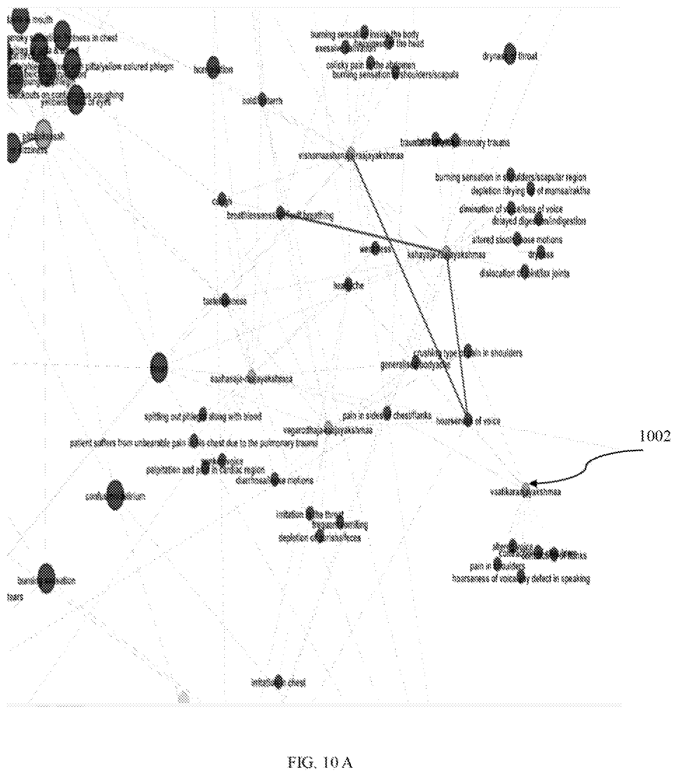

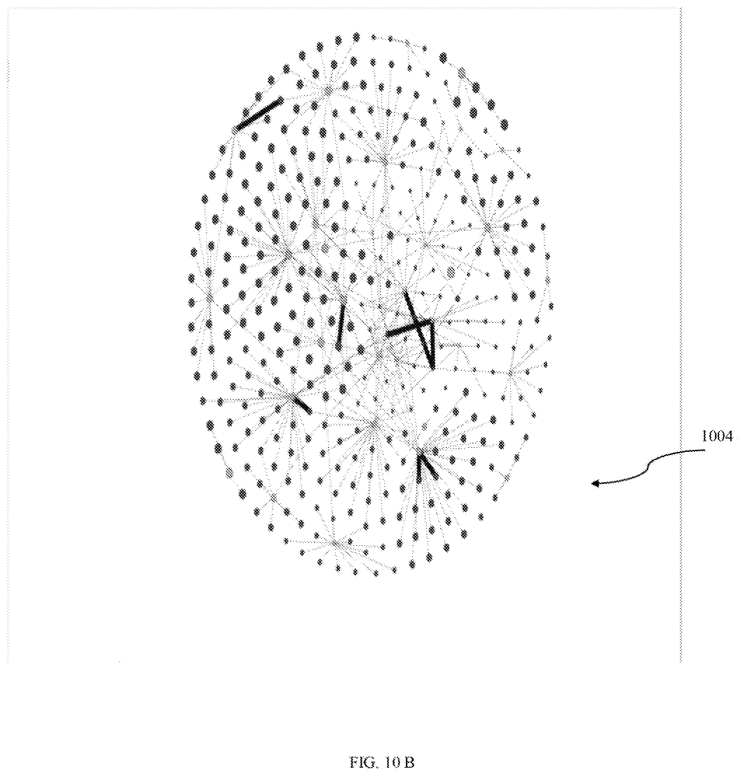

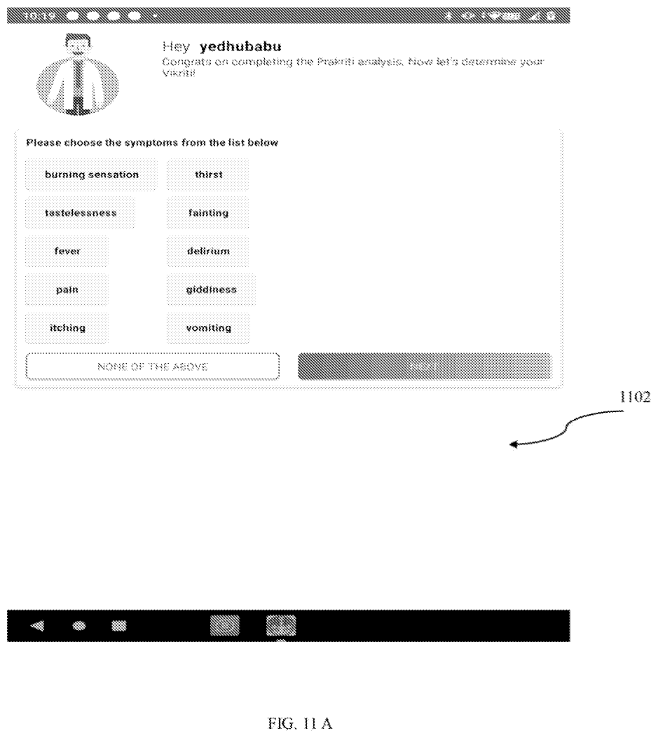

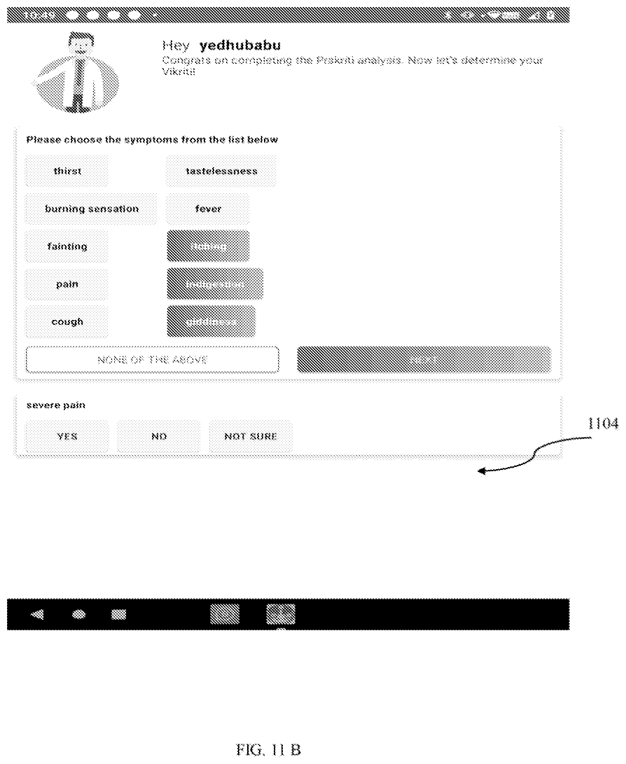

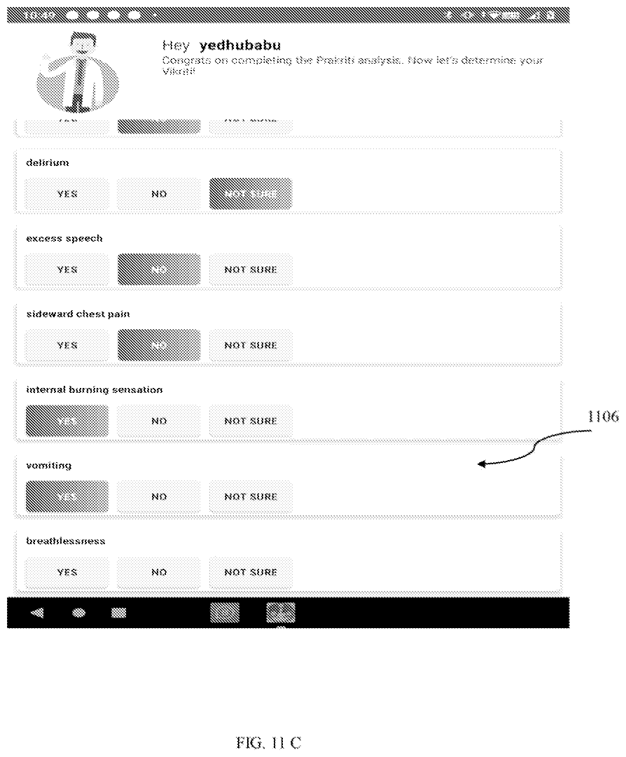

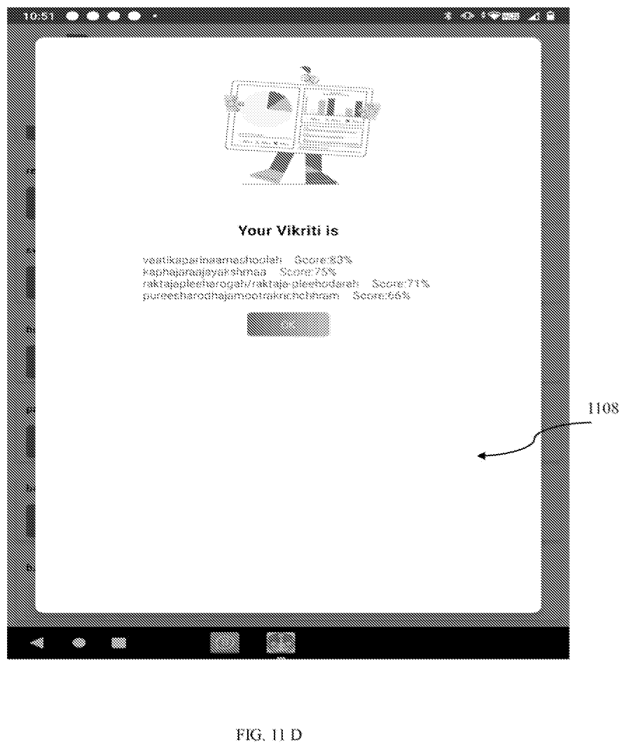

FIG. 10A-B are snapshot representations 1002 and 1004 of weights of the associations between each symptom to the disease as trained using artificial intelligence techniques such as machine learning, probabilistic reasoning, graph neural networks in accordance with an embodiment of the present disclosure. First a symptom-disease association network is built based on Ayurveda principles that relates symptoms to diseases. The weights of the associations 1002 between each symptom to the disease are trained using artificial intelligence techniques using techniques such as machine learning, probabilistic reasoning, graph neural networks and the like. Snapshots of the symptom-disease association network demonstrates the relation between symptoms and diseases

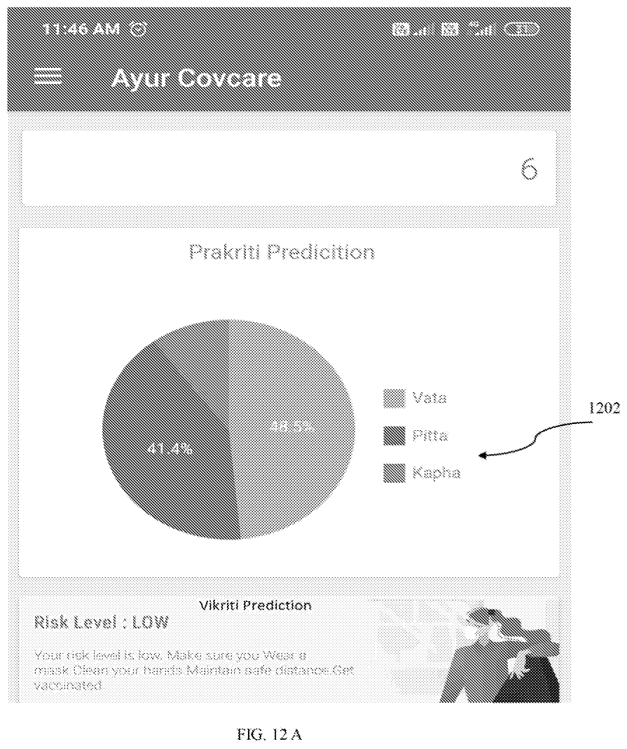

FIG. 11A-D are graphical user interface 1102, 1104, 1106 and 1108 of conversational artificial intelligence-based adaptive questionnaire framework designed as per Ayurveda principles for assessing vikriti (disease) of a person, in accordance with an embodiment of the present disclosure. Subsequently, the patient is presented with various symptoms in an adaptive questionnaire form and depending on the patient responses the network triages the most probable set of diseases. In addition, disease sub-types as described in Ayurveda are also predicted.

In addition, the network also bas explainable artificial intelligence built into the framework so that the disease probabilities based on the selected symptoms is also displayed which aids doctors in making diagnostic decisions. In addition, the explainable artificial intelligence framework also assesses the importance of each of the feature contributing to the vikriti decision. Severity of symptoms can also be asked through the adaptive questionnaire. The disease prediction probability is presented as a percentage or a score. In future, causation-disease, causation-symptom networks may be used to precisely understand the causes of the diseases/symptoms and to design a recommendation engine.

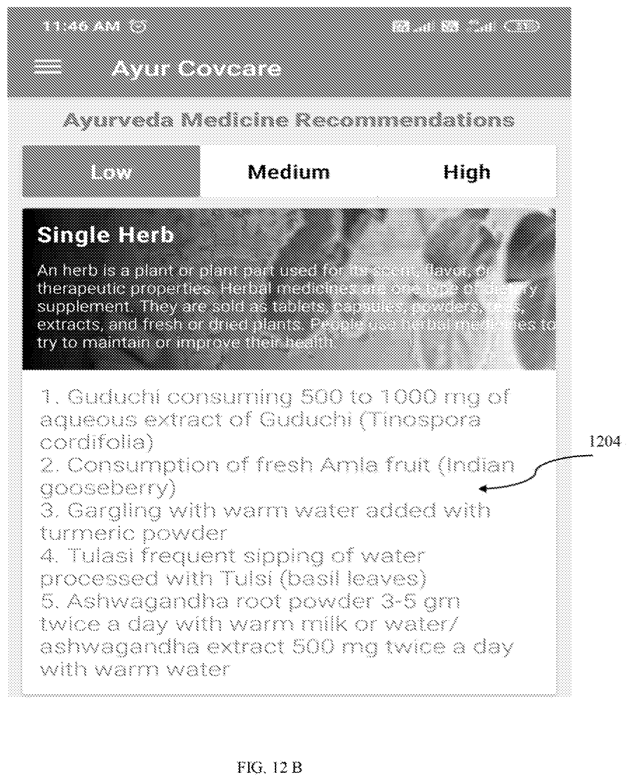

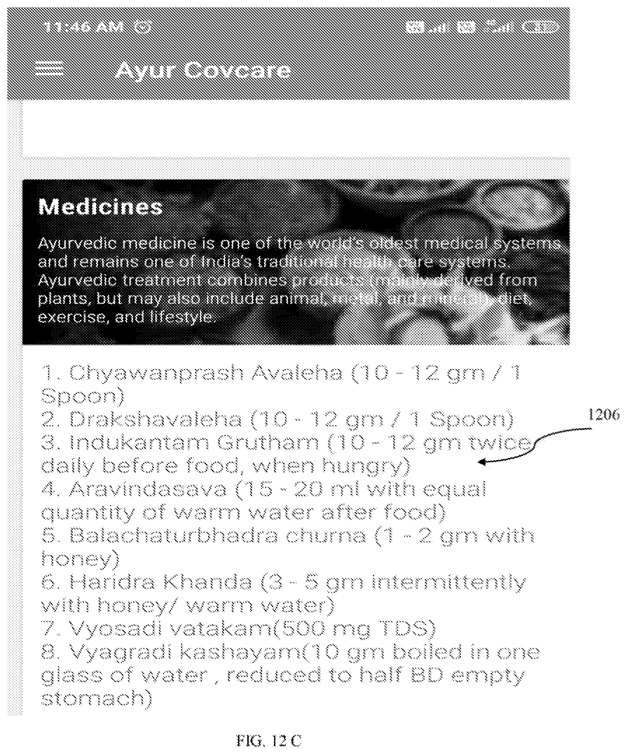

FIG. 12A-C are graphical user interface 1202, 1204 and 1206 of conversational artificial intelligence-based representation for Ayurveda recommendation regarding infectious diseases such as COVID based on the prakriti and vikriti assessment in accordance with an embodiment of the present disclosure. Disease symptoms are captured using a questionnaire. The digital biomarkers are captured from the pulse and the biochemical markers are from the COVID test reports. Digital biomarkers from the pulse such as pulse rate, pulse rate variability are extracted using signal processing algorithms. For example, peaks of the pulse are first extracted and pulse rate as a function of time is calculated. From the peaks, pulse rate variability using various time domain, frequency domain, geometric and nonlinear methods are evaluated for a particular time duration. In addition, digital biomarkers from pulse morphology/shape are extracted from deep learning algorithms. All these features are combined and applied onto a trained machine learning algorithm to predict COVID risk score. Based on the prakriti and the disease risk, the artificial intelligence-based engine gives personalized recommendations. Low, Medium and high risk categorization has different recommendations. The artificial intelligence-based engine provides list of food recommendations as well as medicine recommendations.

Various embodiments of the present disclosure combines both traditional medicine and modern methods for providing highly personalized preventive medicine using quantifiable and data driven approaches. The present disclosure combines multiple inputs from various sources such as adaptive questionnaire, conversational artificial intelligence, computer vision techniques, static and dynamic digital biomarkers, chemical biomarkers, multi-omics markers and uses machine learning and deep learning techniques for quantifiable and personalized assessment of health, wellness and disease.

The knowledge from the baseline and baseline changes may be used for subsequent interventions such as food, medicine, meditation, yoga, panchakarma, acupuncture, music, massage, and the like and to effectively monitor the effectiveness of such interventions. Integrative approach disclosed by the present disclosure can be used to complement the existing allopathy-based diagnosis and interventions. Based on the quantified health, wellness and disease risk parameters, interventions are prescribed, and the quantified parameters are continuously monitored to assess the effectiveness of the intervention so that the interventions can be tailored.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (as shown in FIG. 1) (including but not limited to keyboards, displays, pointing devices, various physiological sensors, electronic and optical sensors etc.) can be coupled to the system either directly or through intervening I/O) controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus to various devices such as a random-access memory (RAM), read-only memory (ROM), and an input/output (I/O) adapter. The I/O adapter can connect to peripheral devices, such as disk units and tape drives, or other program storage devices that are readable by the system. In addition, the system may also comprise of Graphical Processing Unit (GPU) or Tensor Processing Unit (TPU) for running machine and (or) deep learning algorithms. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. An integrative system (100) for performing medical diagnosis using artificial intelligence, the integrative system (100) comprising:
a hardware processor (108); and
a memory (102) coupled to the hardware processor (108), wherein the memory (102) comprises a set of program instructions in the form of a plurality of subsystems, configured to be executed by the hardware processor (108), wherein the plurality of subsystems comprises:
a medical input data collection subsystem (110) configured to collect patient information and phenotypic features associated with a patient from a plurality of medical devices, computer vision techniques, and from a conversational artificial intelligence questionnaire, wherein the phenotypic features comprise anatomic features, physical, physiological features, psychological features;
a baseline health computation subsystem (112) configured to:
apply the collected patient information and phenotypic features associated with the patient on to a trained machine learning model;
generate baseline health status of the patient based on the results of the application of the trained machine learning model, wherein the generated baseline health status comprises a set of integrative medicine system parameters, wherein the set of integrative medicine system parameters comprise a set of traditional medicine system and a set of modern medicine system parameters of the patient;
a baseline change determination subsystem (114) configured to:
obtain a combination of: responses for a set of adaptive and conversational artificial intelligence-based questionnaire in real time from the patient; one or more real time patient information from the plurality of medical devices associated with the patient, one or more real time spatiotemporal information data associated with the patient, and one or more real time inputs from biochemical markers, clinical markers and multi-omics markers;
estimate real time set of integrative medicine system parameters by applying the obtained responses for the set of adaptive and conversational artificial intelligence-based questionnaire, the obtained one or more real time patient information, the obtained one or more real time spatiotemporal information data and the obtained one or more real time inputs from biochemical markers, clinical markers and multi-omics markers onto the trained machine learning model;
determine changes in the generated baseline health status of the patient by comparing the estimated real time set of integrative medicine system parameters with the estimated set of integrative medicine system parameters associated with the generated baseline health status by using artificial intelligence-based comparison model; and
a health, wellness, and disease risk score computation subsystem (116) configured to:
generate a health, wellness, and disease risk score of the patient based on determined changes in the generated baseline health status of the patient; and
generate a recommendation message to the patient based on the generated health, wellness, and disease risk score.

2. The integrative system (100) as claimed in claim 1, wherein the phenotypic features of the patient are obtained using plurality of physiological sensors, computer vision and voice-based techniques and various biochemical, clinical and multi-omics markers.

3. The integrative system (100) as claimed in claim 1, wherein the phenotypic feature comprises digital markers extracted from pulse electrocardiography (ECG) data, photo plethysmograph (PPG) data, electroencephalogram (EEG) data, bioimpedance sensor data, galvanic skin response data, multispectral reflectance data, transmittance data, and autofluorescence data from plurality of body parts, sleep activity data, physical activity data and mental activity data.

4. The integrative system (100) as claimed in claim 1, wherein the one or more real time spatiotemporal information data associated with the patient comprises health parameters of the patient with respect to a location and a period of time.

5. The integrative system (100) as claimed in claim 1, wherein the recommendation message comprises of medical diagnosis of the disease, health parameters, therapeutic interventions, clinical interventions, one or more medical remedies, and treatment plan.

6. An integrative method (700) for performing medical diagnosis using artificial intelligence, the integrative method (700) comprising:
collecting, by a processor (108), patient information and phenotypic features associated with a patient from a plurality of medical devices, computer vision techniques, and from a conversational artificial intelligence questionnaire (702), wherein the phenotypic features comprise anatomic features, physical, physiological features, psychological features;
applying, by the processor (108), the collected patient information and phenotypic features associated with the patient on to a trained machine learning model (704);
generating, by the processor (108), baseline health status of the patient based on the results of the application of the trained machine learning model (706), wherein the generated baseline health status comprises a set of integrative medicine system parameters of the patient, wherein the set of integrative medicine system parameters comprise a set of traditional medicine system and a set of modern medicine system parameters of the patient;
collecting, by the processor (108), obtain a combination of: responses for a set of adaptive and conversational artificial intelligence-based questionnaire in real time from the patient, one or more real time patient information from the plurality of medical devices associated with the patient, one or more real time spatiotemporal information data associated with the patient, and one or more real time inputs from biochemical markers and multi-omics markers (708);
estimating, by the processor (108), real time set of integrative medicine system parameters variables by applying the obtained responses for the set of adaptive and conversational artificial intelligence-based questionnaire, the obtained one or more real time patient information, the obtained one or more real time spatiotemporal information data and the obtained one or more real time inputs from biochemical markers, clinical markers and multi-omics markers onto the trained machine learning model (710);
determining, by the processor (108), changes in the generated baseline health status of the patient by comparing the estimated real time set of integrative medicine system parameters with the estimated set of integrative medicine system parameters associated with the generated baseline health status by using artificial intelligence-based comparison model (712);
generating, by the processor (108), a health, wellness, and disease risk score of the patient based on determined changes in the generated baseline health status of the patient (714); and
generating, by the processor (108), a recommendation message to the patient based on the generated health, wellness, and disease risk score (716).

7. The integrative method (700) as claimed in claim 6, wherein the phenotypic features comprise digital markers extracted from electrocardiography (ECG) data, photo plethysmograph (PPG) data, electroencephalogram (EEG) data, bioimpedance sensor data, galvanic skin response data, multispectral reflectance data, transmittance data, and autofluorescence data from various body parts, sleep activity data, physical activity data and mental activity data.

8. The integrative method (700) as claimed in claim 6, wherein the phenotypic feature of the patient comprises obtaining features using plurality of physiological sensors, voice-based techniques and various biochemical, clinical and multi-omics markers.

9. The integrative method (700) as claimed in claim 6, wherein the one or more real time spatiotemporal information data associated with the patient comprises health parameters of the patient with respect to a location and a period of time.

10. The integrative method (700) as claimed in claim 6, the recommendation message comprises medical diagnosis of the disease, health parameters, therapeutic interventions, clinical interventions, one or more medical remedies, and treatment plan.
